(19) United States
(12) Patent Application Publication
(10) Pub. No.: US 2009/0163930 A1
(43) Pub. Date: Jun. 25, 2009

(54) CALIBRATION SYSTEM OF A COMPUTER-ASSISTED SURGERY SYSTEM

(76) Inventors: Ahmed Aoude, Pointe-Claire (CA); Bruno Falardeau, Verdun (CA); Melanie Chasse, Deux-Montagnes (CA); Karine Duval, Montreal (CA)

Correspondence Address: OGILVY RENAULT LLP, 1981 MCGILL COLLEGE AVENUE, SUITE 1600, MONTREAL, QC, H3A2Y3, CA

(21) Appl. No.: 12/339,405

(22) Filed: Dec. 19, 2008

Related U.S. Application Data
(60) Provisional application No. 61/014,924, filed on Dec. 19, 2007.

(51) Int. Cl. A61B 19/00

(52) U.S. Cl. 606/130

(57) ABSTRACT

A calibration system for the calibration of a surgical instrument, comprises a surgical instrument having a working shaft. A clamp has a first trackable member and is releasably connected to the surgical instrument such that an orientation relation between the clamp and the working shaft of the instrument is known. A base is separated from the clamp and has a second trackable member and a positioning portion being tracked for position. A sensor unit tracks the first and second trackable members. A calibration unit calibrates the surgical instrument by initializing the orientation relation between the clamp and the working shaft from the tracking data to obtain an orientation of the working shaft, and by setting a position for the working shaft with respect to the first trackable member by relating the orientation of the working shaft to the position of the positioning portion when the working shaft contacts the positioning portion.

### CROSS-REFERENCE TO RELATED APPLICATION

This patent application claims priority on U.S. Provisional Patent Application No. 61/014,924, filed on Dec. 19, 2007.

### FIELD OF THE APPLICATION

The present application relates to computer-assisted surgery instrumentation and, more particularly, to the calibration thereof.

### BACKGROUND OF THE ART

In computer-aided surgery, it is known to use surgical instruments detectable by positioning systems in order to have an on-screen representation of the instrument with respect to an operated part of a patient's body. It is readily understood that great amounts of precision and accuracy are required in the space positioning of the surgical instruments in order to obtain reliable representation of the operation. A misrepresentation of the instrument with respect to the patient's body may have dramatic consequences and may even be fatal to the patient. Thus, prior to computer-aided surgery, the instruments must be calibrated.

One known method of calibrating is referred to as the axial-conical calibration. This method consists in achieving predetermined maneuvers with a surgical instrument having detectable devices thereon for it to be located in space by sensors connected to a position calculator. Namely, a first maneuver consists in rotating the surgical instrument with respect to its longitudinal axis, whereby the position of the latter is set. During this rotation, the position calculator receives readings which will allow it to calculate a transform matrix from the positioning system to the axis of the instrument. Thereafter, in a second maneuver, the instrument is rotated according to a conical trajectory having as an apex the working tip thereof. Hence, the positioning system may interpret and find another transform matrix between the positioning system and the tip of the surgical instrument. Although the axial-conical calibration method is simple, the required maneuvers of calibration may take a few minutes to an inexperienced user and the position calculator may require the maneuvers to be repeated if they are judged as being unsatisfactory.

### SUMMARY OF THE APPLICATION

It is therefore an aim of the present application to provide a novel method and system for automatically calibrating surgical instruments.

Therefore, in accordance with the present application, there is provided a calibration system of a computer-assisted surgery system for the calibration of a surgical instrument, comprising: a surgical instrument having a working shaft; a clamp having a first trackable member and being releasably connected to the surgical instrument such that an orientation relation between the clamp and the working shaft of the instrument is known; a base separated from the clamp having a second trackable member and having a positioning portion being tracked for position; a sensor unit for tracking the first and second trackable members; a calibration unit for calibrating the surgical instrument by initializing said orientation relation between the clamp and the working shaft from the tracking of the first trackable member to obtain an orientation of the working shaft, and by setting a position for the working shaft with respect to the first trackable member by relating the orientation of the working shaft to the position of the positioning portion when the working shaft contacts the positioning portion.

Further in accordance with the present application, there is provided a trackable device for a surgical instrument used in computer-assisted surgery comprising: a trackable member; a clamp supporting the trackable member and having a trough adapted to abut against a working shaft of the surgical instrument away from a tip of the surgical instrument such that an inner edge of the trough is parallel to an axis of the working shaft, the clamp being releasably connected to the working shaft of the surgical instrument such that an orientation relation between the clamp and the working shaft of the instrument is known for subsequently tracking the surgical instrument.

Further in accordance with the present application, there is provided use of an optical trackable member having a body supporting at least three trackable elements at ends thereof as a calibration base by knowing a position of a connection portion with respect to a geometrical pattern formed by the at least three trackable elements.

### BRIEF DESCRIPTION OF THE DRAWINGS

FIG. 1 is a perspective view of a tracked surgical instrument used with a calibration clamp and a calibration base in accordance with an embodiment of the present application; and

FIG. 2 is a calibration system of a computer-assisted surgery system in accordance with another embodiment of the present application.

FIG. 3 is a perspective view of a surgical instrument without tracker used with a calibration clamp and a calibration base in accordance with another embodiment of the present application;

FIG. 4 is a perspective view of a calibration base in accordance with another embodiment of the present application;

FIG. 5 is another perspective view of a surgical instrument used with a calibration clamp and a stopper on the shaft of the surgical instrument; and

FIG. 6 is a perspective view of an untracked surgical instrument with a calibration clamp and a calibration base similar to that of FIG. 4.

### DESCRIPTION OF THE PREFERRED EMBODIMENTS

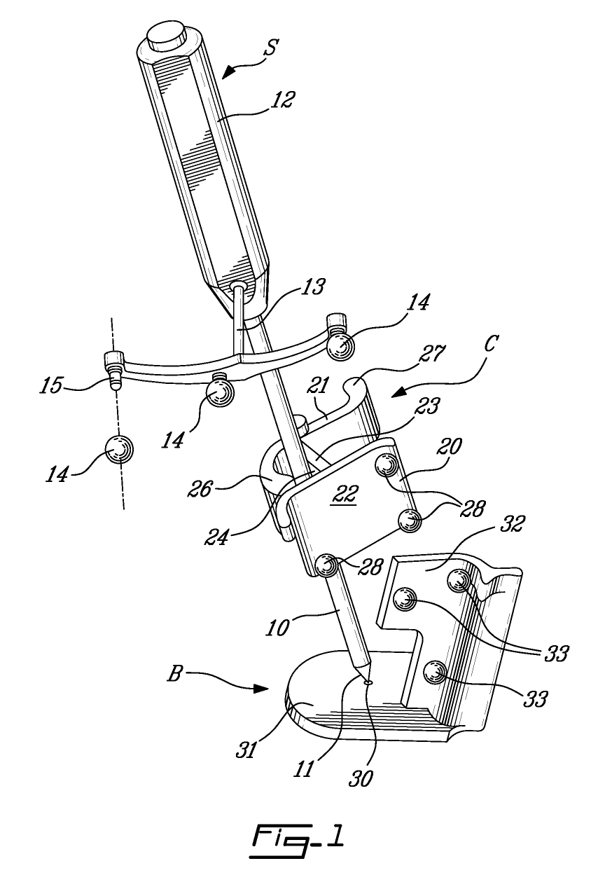

Referring to the drawings and more particularly to FIG. 1, a surgical instrument S is shown in position with respect to a calibration clamp C and a calibration base B in accordance with an embodiment of the present application. The instrument S, clamp C and base B are part of a calibration system of a CAS system. The clamp C and the base B are provided to define a position and an orientation for the instrument S.

In the illustrated embodiment, the surgical instrument S is a registration pointer having a shaft 10 of circular cross-section, although other cross-sections are considered as well. A tip 11 is disposed at a working end of the shaft 10, whereas a handle 12 is disposed at a handling end of the instrument S. A support 13 extends from the handle 12 of the surgical instrument S. The support 13 supports a trackable member, herein a first set of detectable spheres 14.

The detectable spheres 14 are coated with a retro-reflective layer in order to be detected by, for instance, an infrared sensor using axial illumination. It is pointed out that other shapes are known and could also be used as alternative embodiments to retroreflective spheres. As an example, straight cylinders, corner reflectors or the like having retroreflective properties could also be used.

It is also pointed out that the detectable spheres 14 may be removed by providing snap-fit mating adapters, such as the one illustrated by 15, such that single-use spheres may be used. This allows for the spheres to be sterilized with processes milder than autoclave sterilization, whereby a coating does not need to be characterized by its capability to sustain high temperatures or pressures. Moreover, retroreflective tokens and like detectable members can be used as alternatives to the spheres.

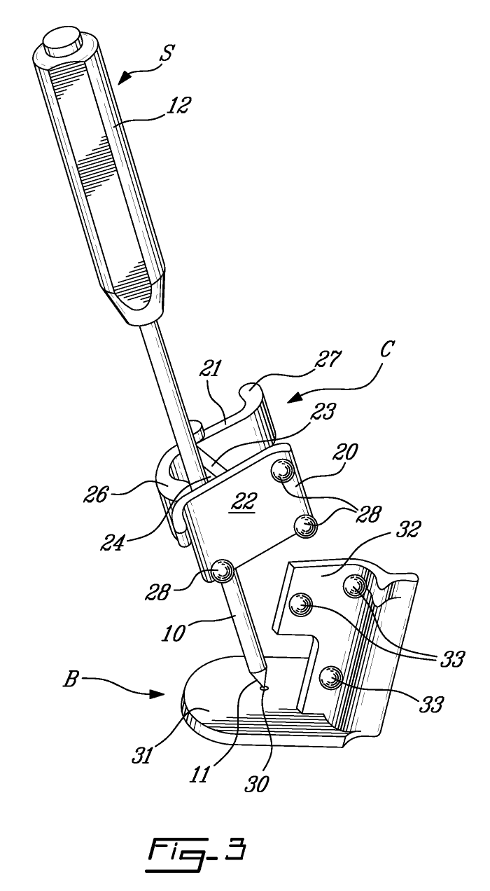

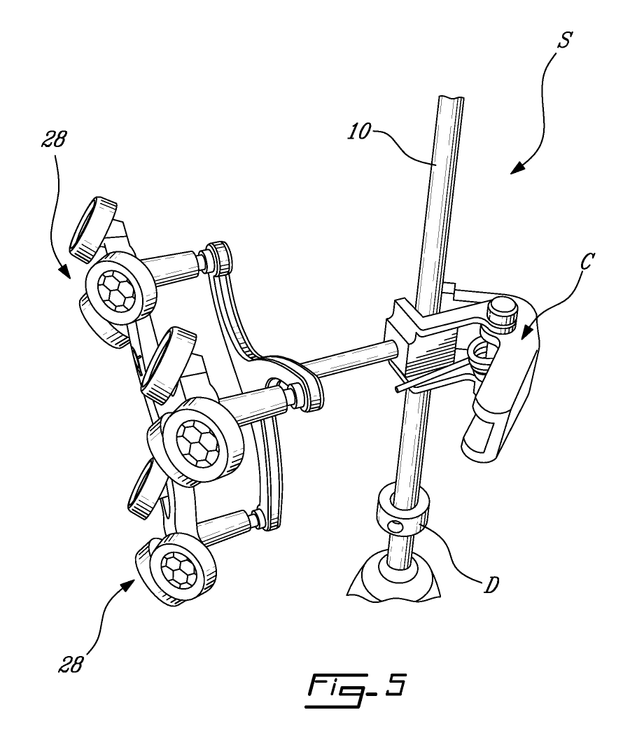

It is pointed out that the surgical instrument S need not be provided with a trackable member to be calibrated and subsequently tracked. As shown in FIG. 3, the untracked surgical instrument S is used with a calibration base B and a calibration clamp C. The calibration clamp C can be used for the tracking of the surgical instrument S instead of the trackable member 14. In such a case, the clamp C is advantageously used as a temporary trackable element. It is however pointed out that the clamp C must remain in a fixed position along the instrument S to be used as a trackable element. Therefore, as is shown in FIG. 5, a stopper D is optionally provided on the shaft 10 of the surgical instrument S to limit axial movement of the clamp C along the shaft 10. In FIG. 5, the clamp C is separated from the stopper D for clearly illustrating the stopper D on the shaft 10. It is however pointed out that the clamp C contacts the stopper D if a stopper D is used. The stopper D is manually installed on the shaft 10 of the instrument S. For instance, the clamp C may be abutted against the handle of the instrument S, a shoulder on the shaft, etc.

Referring to FIG. 1, the calibration clamp C is releasably secured to the shaft 10 of the instrument S, in a known orientation relation, as will be described hereinafter. The calibration clamp C has an abutment member 20 and a lever 21.

The abutment member 20 is trough-shaped, and features walls 22 and 23. The walls 22 and 23 are perpendicular to one another, so as to define a right-angle abutment corner 24. Although the illustrated embodiment discloses them in a perpendicular relation, the walls 22 and 23 may be in any relation with respect one to another, although it is preferred that the shaft 10 be parallel to the walls 22 and 23 when abutted against same. For instance, the walls 22 and 23 may define a V-shaped channel of obtuse or acute angles for receiving the working shaft 10 thereagainst, even though the illustrated embodiment discloses a right angle therebetween.

The lever 21 is pivotally mounted to the wall 23, and has rounded ends 26 and 27 at its extremities. A spring biases the lever 21 such that the rounded end 26 is biased toward the abutment corner 24. The lever 21 consists of a material able to sustain the high pressure of an autoclave during the sterilizing of the calibration clamp C, such as an acetal copolymer.

Accordingly, as is shown in FIG. 1, the lever 21 clamps the working shaft 10 of the instrument S in the abutment corner 24. The resilient forces of the spring of the lever 21 are such that the clamp C remains in position along the shaft 10, while a manual actuation is sufficient to release the clamp C from the shaft 10. It is pointed out that alternative mechanisms may be used instead of the spring-biased lever 21, so long as the working shaft is pressured against the walls 22 and 23.

It is observed that the shaft 10 is elongated and has a uniform cross-section. Therefore, the edge along the abutment corner 24 is parallel to the longitudinal axis of the shaft 10 when the clamp C is secured to the shaft 10.

The wall 22 supports another trackable member, herein another set of detectable spheres 28. The detectable spheres 28 are similar in construction to the above mentioned detectable spheres 14 (in the event that the surgical instrument S has a trackable member). The detectable spheres 28 are tracked, whereby the position and orientation of the calibration clamp C is calculable.

Referring to FIG. 1, the calibration base B has a positioning receptacle 30 (i.e., a connection or positioning portion) in base member 31. A wall member 32 is connected to the base member 31, and supports a trackable member, herein a third set of detectable spheres 33. The detectable spheres 33 are fixed to the wall member 32, whereby the spatial relation between the spheres 33 and the positioning receptacle 30 is fixed. Therefore, the connection portion/positioning receptacle 30 is trackable for position by the tracking of the spheres 33.

The positioning receptacle 30 is shaped so as to accommodate the tip 11 of the instrument S, whereby the tip 11 in the receptacle 30 is in a known position.

The calibration clamp C can be used with surgical instruments having working shafts of a wide range of cross-section shapes and diameters (e.g. 3 to 37 mm). Although a registration pointer having a pointy end is illustrated, other embodiments are considered as well. If the cross section of the shaft 10 of the instrument S is circular, the clamp C may rotate about the shaft 10 while the position of the tip 11 is tracked, as long as the position of the clamp C along the shaft 10 does not change (whereby a stopper D is preferred in such cases). This feature advantageously allows a screwing axis of a screw-driving tool to be tracked, as it is possible to maintain a line of sight between the detectable spheres 28 of the clamp C and a sensor unit, by allowing a rotation of the instrument S while the clamp C remains oriented toward the sensor unit.

It is pointed out that the instrument S, the clamp C and the base B are provided with optically detectable members for passive tracking. However, it is pointed out that active tracking members (e.g., electromagnetic, RF) may be used as an alternative to the optical tracking system.

In order to track simultaneously the three sets of detectable spheres, it is pointed out that each set of spheres (i.e., 14, 28 and 33) is in a different scalene triangular arrangement.

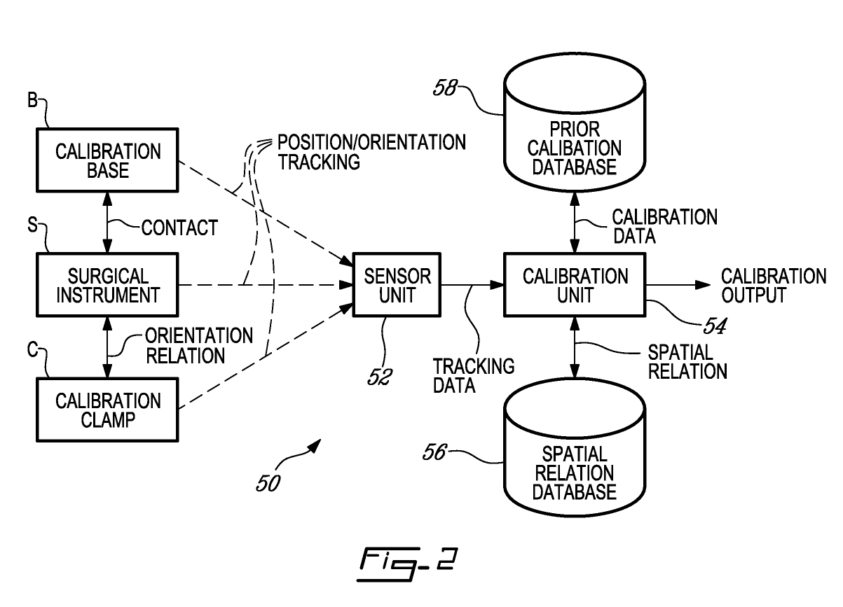

Referring to FIG. 2, a calibration system that is part of a computer-assisted surgery system is generally shown at 50. The calibration system 50 is used to calibrate the surgical instruments, such as the instrument S, for their subsequent use in computer-assisted surgery.

The calibration system 50 comprises the calibration base B and the calibration clamp C. The calibration system 50 has a sensor unit 52 so as to track the detectable members of the base B, the clamp C and the instrument S. The nature of the sensor unit 52 is dependent on the type of detection system used. In the illustrated embodiments, the sensor unit 52 tracks the detectable spheres 14, 28 and 33 for position.

A calibration unit 54 is connected to the sensor unit 52 so as to receive the tracking data. In an embodiment, the calibration unit 54 is a computer program part of the processing unit of a computer-assisted surgery system. The calibration unit 54 calibrates the surgical instrument S from the tracking data provided by the sensor unit 52 and by data provided by the spatial relation database 56 and/or the prior calibration database 58.

The spatial relation database 56 stores the triangular arrangements of each set of detectable spheres 28 and 33, and 14 if applicable. Therefore, by providing the triangular arrangements to the calibration unit 54, the calibration unit 54 may identify and individually track the base B, the clamp C and the surgical instrument S (if applicable).

The database 56 may also store data pertaining to the geometry of the base B and clamp C. For instance, the spatial relation between the positioning receptacle 30 (i.e., the connection portion) and the triangular arrangement of the trackable member 33 is known, whereby the position of the positioning receptacle 30 is tracked by the calibration unit 54 as the base B is tracked.

Similarly, the database 56 stores a spatial relation between the edge of the abutment corner 24 and the triangular arrangement of detectable spheres 28. As discussed previously, the edge of the abutment corner 24 is parallel to the working shaft 10 of the instrument S when the shaft 10 is clamped in the abutment corner 24. Therefore, the spatial relation between the axis passing through the edge and the triangular arrangement of detectable spheres 28 is provided to the calibration unit 54 by the database 56, whereby the axis is tracked by the calibration unit 54.

Therefore, in accordance with a step of the calibration of the instrument S, when the calibration clamp C is secured to the surgical instrument S, the calibration unit 54 knows the relation between the clamp C and the shaft 10 (e.g., in the present embodiment, the shaft 10 is parallel to the edge of the abutment corner 24). The calibration unit 54 can therefore initialize the relation between the clamp C and the shaft 10 to set an orientation for the shaft 10 with respect to the trackable member (1) on the clamp C, namely the detectable spheres 28 for the embodiment of FIG. 3, or (2) on the surgical instrument S, namely the detectable spheres 14, for the embodiment of FIG. 1. Therefore, after this calibration step, an orientation has been set by the calibration unit 54 for the working shaft 10 of the surgical instrument S, and is tracked as a function of the clamp C on the instrument S (embodiment of FIG. 3) or of the trackable member 14 of the instrument S (embodiment of FIG. 1).

In accordance with another step of calibration, the tip 11 of the shaft 10 is positioned in the positioning receptacle 30 in a known contacting relation. As the position of the positioning receptacle 30 is known and tracked, the calibration unit 54 sets the position of the tip 11 of the working shaft 10 with respect to the trackable member 14 (embodiment of FIG. 1) or 28 (embodiment of FIG. 3) when the surgical instrument S contacts the base B.

A position and orientation of the surgical instrument S with respect to the trackable member 28 or its trackable member 14 has therefore been established by the calibration unit 54. Namely, the position and orientation of the working shaft 10 of the instrument S is an axis that is parallel to the edge of the abutment corner 24 of the clamp C, which axis passes through the position of the positioning receptacle 30 of the base B when the tip 11 contacts the positioning receptacle 30. The position and orientation of the surgical instrument S with respect to trackable member 28 or its trackable member 14 is the calibration output from the calibration unit 54 and is used by the computer-assisted surgery system during surgery for the tracking of the surgical instrument S.

A prior calibration database 58 is also provided, as an option. For instance, it may be required to calibrate the base B or the clamp C prior to calibrating the surgical instrument S. For instance, the clamp C may be calibrated by obtaining a normal to the plane of the T-shaped surface formed by the walls 22 and 23. However, it is preferred that the calibration base B and clamp C be permanently calibrated.

The database 58 also stores the prior calibration data, which consists of the calculated position and orientation of the tips of all the surgical instruments which were calibrated previously. This allows for a validation of the calibration of the instruments. For instance, the instrument S depicted in FIG. 1 is calibrated and used for surgery. The calibration unit 54 will automatically store the position and orientation of the working shaft 10 of the instrument S with respect to the trackable member thereon, namely the spheres 14 and/or 28 in the triangular arrangement.

Thereafter, at the next calibration of the same instrument S, the calibration unit 54 will optionally compare the new calculated position and orientation of the working shaft 10 to the reference position and orientation stored in the database 58. If the new calculated position and orientation are not within an allowable range, the operator will be prompted to verify the state of the instrument S and of the base B and clamp C. If, after a second reading of the sensors 52, the position and orientation are the same as the previous one, the operator will be prompted to either accept the new space position and orientation, or to retry calibrating until the stored reference position and orientation are attained.

In order to ensure the precision of the calibration, it is considered to store in the spatial relation database 56 a range of distances by which the calibrated axis of the shaft 10 is offset from the edge of the abutment corner 24 of the clamp C.

Therefore, when the tip 11 is in the positioning receptacle 30, the axis of the edge of the abutment corner 24, which has been registered with respect to the trackable member 14 or 28, must be offset by a specific value to pass through the positioning receptacle 30. If the specific value falls outside of the range of distances, the calibration unit 54 may indicate to the operator that the calibration may be faulty. The range of distances is established knowing the diameters of the surgical instruments that will be calibrated with the system 50.

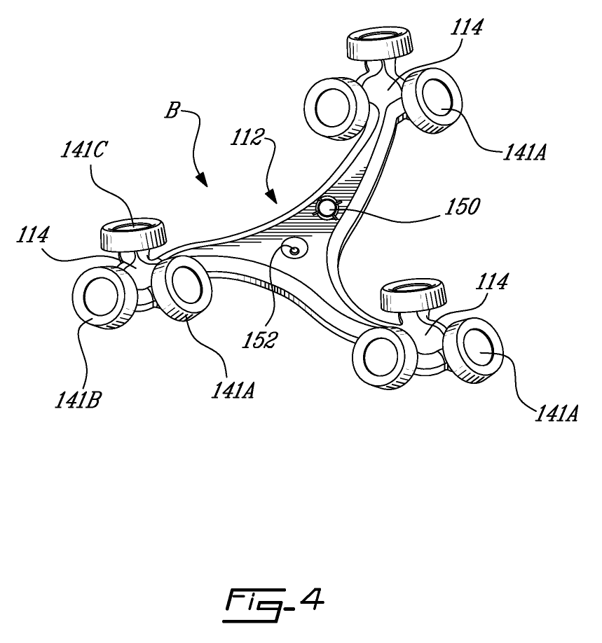

Referring to FIG. 4, the calibration base B in accordance with another embodiment is generally shown as a multifaceted tracker device similar to the one of United States Publication No. 2007/0100325 (by Jutras et al. as published on May 3, 2007). If the multifaceted tracker device described above is used as the calibration base B, calibrating or the like must be done to ensure that the multifaceted tracker device provides suitable results. For instance, it may be required to connect the multifaceted tracker device to a structure to ensure it does not bend as a result from contact from the instrument S. The tracker device 10 has a support 112 provided to interrelate the tracker device to a surgical tool (e.g., registration pointer, rasp, drill guide, reference base or like instruments used in CAS). The support 112 supports the tracker ends 114 in a given geometry, such that an optical sensor unit 52 of a CAS system visually recognizes the patterns. With the tracking of the patterns of the tracker ends 114, the CAS system calculates a position and/or orientation of the surgical instrument or tool associated with the tracker device.

Accordingly, in the embodiment of FIG. 4, the three tracker ends 114 are provided in three sets of three detectable elements. The tracker ends 114 are each provided with faces 141A, 141B and 141C (hereinafter faces 141 unless otherwise indicated). The faces 141 each define an opening having a given geometrical shape. In the embodiment of FIG. 4, the given geometrical shape is a circle.

Retro-reflective surfaces are positioned in the openings, so as to form circular optical elements provided in the faces 141 of the tracker ends 114. Other shapes are also considered for the optical elements. The retro-reflective surfaces are made of a retro-reflective material that will be detected by the optical sensor apparatus associated with the CAS system. For instance, the material Scotch-Lite™ is suited to be used as retro-reflective surface. Female connectors (not shown) are provided in an underside of the support 112 and are used to connect the support 112 to a surgical instrument.

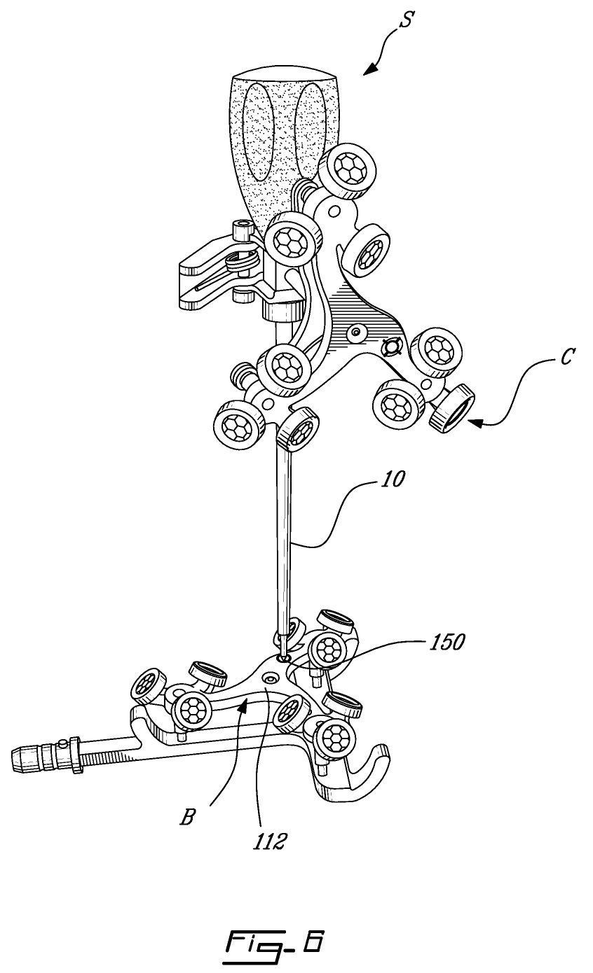

As is shown in FIG. 4, a positioning receptacle 150 is provided on the front face of the support 112. The position of the positioning receptacle 150 is known with respect to the geometrical pattern defined by the faces 141, whereby the tip 11 of the instrument S can be inserted in the receptacle 150 for the position calibration of the tip 11, as described previously for FIGS. 1 to 3. In FIG. 6, the tip 11 of the surgical instrument S is inserted in the positioning receptacle 150 to be calibrated. There may be more than one positioning receptacle 150 on the support 112. In such a case, each has its own position known with respect to the geometrical pattern defined by the faces 141. Moreover, the range of distances is established knowing the orientation of the clamp C as well as the range of diameters of the surgical instruments that will be calibrated with the calibration base of FIG. 4, so as to indicate faulty calibration if other parts of the calibration base of FIG. 4 are used, such as the injection point 152.

Referring to FIG. 6, the shaft 10 of the surgical instrument S is close to being normal to a plane of the calibration base B, the plane passing through the support 112. It is preferred that the shaft 10 be close to normal to the plane of the calibration base B during calibration to ensure the accuracy of the calibration. Accordingly, the calibration system 50 may measure the angle at which the shaft 10 is with respect to the normal of the plane, and refuse to calibrate if the angle is above a predetermined limit (e.g., 60° from the normal). In such a case, the calibration system 50 prompts the user to repeat the calibration with the shaft S being closer to the normal of the plane of the calibration base B.

1. A calibration system of a computer-assisted surgery system for the calibration of a surgical instrument, comprising:
a surgical instrument having a working shaft;
a clamp having a first trackable member and being releasably connected to the surgical instrument such that an orientation relation between the clamp and the working shaft of the instrument is known;
a base separated from the clamp having a second trackable member and having a positioning portion being tracked for position;
a sensor unit for tracking the first and second trackable members;
a calibration unit for calibrating the surgical instrument by initializing said orientation relation between the clamp and the working shaft from the tracking of the first trackable member to obtain an orientation of the working shaft, and by setting a position for the working shaft with respect to the first trackable member by relating the orientation of the working shaft to the position of the positioning portion when the working shaft contacts the positioning portion.

2. The calibration system according to claim 1, wherein the clamp has a spring-biased lever for being clamped to the working shaft in said orientation relation.

3. The calibration system according to claim 2, wherein the clamp has a trough abutting against the working shaft such that an inner edge of the trough is parallel to an axis of the working shaft.

4. The calibration system according to claim 3, wherein the trough is a right-angle trough.

5. The calibration system according to claim 1, wherein the surgical instrument has a stopper protruding radially from the working shaft so as to block the clamp from sliding along the working shaft.

6. The calibration system according to claim 1, wherein the clamp is used subsequently to calibration for tracking the surgical instrument during surgery.

7. The calibration system according to claim 6, wherein the surgical instrument is used in a screwing motion, with the clamp being maintained in a fixed orientation with respect to the sensor unit and a fixed position with respect to the surgical instrument while the surgical instrument performs the screwing motion.

8. The calibration system according to claim 1, wherein the calibration unit validates the calibration of the surgical instrument by comparing (1) the position and orientation of the working shaft with respect to the claim resulting from the calibration with (2) the position and orientation of the working shaft with respect to the clamp resulting from a previous calibration.

9. The calibration system according to claim 1, wherein the calibration unit validates the calibration of the surgical instrument by comparing (1) the position and orientation of the working shaft with respect to the claim resulting from the calibration with (2) a given range of working shaft diameters.

10. The calibration system according to claim 1, wherein the base is a multi-faceted tracker.

11. A trackable device for a surgical instrument used in computer-assisted surgery comprising:
a trackable member;
a clamp supporting the trackable member and having a trough adapted to abut against a working shaft of the surgical instrument away from a tip of the surgical instrument such that an inner edge of the trough is parallel to an axis of the working shaft, the clamp being releasably connected to the working shaft of the surgical instrument such that an orientation relation between the clamp and the working shaft of the instrument is known for subsequently tracking the surgical instrument.

12. The trackable device according to claim 11, further comprising a spring-biased lever adapted to urge the clamp against the working shaft in said orientation relation.

13. The trackable device according to claim 11, wherein the trough is a right-angle trough.
